(19) United States
(12) Patent Application Publication
(10) Pub. No.: US 2014/0111631 A1
(43) Pub. Date: Apr. 24, 2014

(54) AUXILIARY EQUIPMENT AND METHOD FOR ASSISTING USAGE OF EYEDROPPER

(71) Applicant: HON HAI PRECISION INDUSTRY CO., LTD., New Taipei (TW)

(72) Inventors: HOU-HSIEN LEE, New Taipei (TW); CHANG-JUNG LEE, New Taipei (TW); CHIH-PING LO, New Taipei (TW)

(73) Assignee: HON HAI PRECISION INDUSTRY CO., LTD., New Taipei (TW)

(21) Appl. No.: 14/050,397

(22) Filed: Oct. 10, 2013

(30) Foreign Application Priority Data
Oct. 22, 2012 (TW) 101138937

(51) Int. Cl. A61B 19/00

(52) U.S. Cl. CPC A61B 19/5212
USPC 348/78

(57) ABSTRACT

In a method for assisting usage of an eyedropper using auxiliary equipment, a current image is captured by an image capturing unit of the auxiliary equipment, one or more eyes are detected in the current image, and one or more rectangular areas enclosing the eyes are determined. One or more first specified ranges are determined according to positions of the rectangular areas, the first specified ranges are determined to be proper areas of the eyes for dropping eye drops from the eyedropper. A position of each of the first specified ranges is compared with a position of a second specified range of a tip of the eyedropper, to determine whether the second specified range is overlapping with one of the first specified ranges. An indicator lamp of the auxiliary equipment is activated according to a determination result.

### BACKGROUND

1. Technical Field

Embodiments of the present disclosure relate to assistant technology, and particularly to an auxiliary equipment and a method for assisting with the use of an eyedropper using the auxiliary equipment.

2. Description of Related Art

A tip of an eyedropper coming close to an eye, may cause the user to blink, which may result in the liquid being dropped on top of the eyelids. On the other hand, it is hard to see the eyedropper clearly when a distance between the eyes and the tip is very short because the eye cannot focus on the eyedropper accurately at a short distance. Therefore, a prompt and efficient method for assisting usage of the eyedropper is desired.

### BRIEF DESCRIPTION OF THE DRAWINGS

FIG. 1 is a block diagram of one embodiment of an auxiliary equipment.

FIG. 2 is a schematic diagram of the auxiliary equipment equipped with an image capturing unit and a plurality of indicator lamps.

FIG. 3 is a schematic diagram of the auxiliary equipment connected with a transmitting line.

FIG. 4 is a schematic diagram of the auxiliary equipment holding an eyedropper.

FIG. 5 is a block diagram of function modules of an eyedropper assistant system included in the auxiliary equipment.

FIG. 6 is a flowchart of one embodiment of a method for assisting with the use of the eyedropper using the auxiliary equipment.

FIG. 7 is a schematic diagram of rectangular areas of the eyes detected in a current image captured by the image capturing unit.

FIG. 8 is a schematic diagram of first specified ranges determined according to the positions of the rectangular areas in FIG. 7.

FIG. 9 is a schematic diagram of a second specified range determined according to the current image captured by the image capturing unit.

FIG. 10 is a schematic diagram of activating a first indicator lamp when a tip of the eyedropper is not located in a proper area of the eye.

FIG. 11 is a schematic diagram of activating a second indicator lamp when the tip of the eyedropper is located in the proper area of the eye.

### DETAILED DESCRIPTION

All of the processes described below may be embodied in, and fully automated via, functional code modules executed by one or more general purpose electronic devices or processors. The code modules may be stored in any type of non-transitory computer-readable medium or other storage device. Some or all of the methods may alternatively be embodied in specialized hardware. Depending on the embodiment, the non-transitory computer-readable medium may be a hard disk drive, a compact disc, a digital video disc, a tape drive or other suitable storage medium.

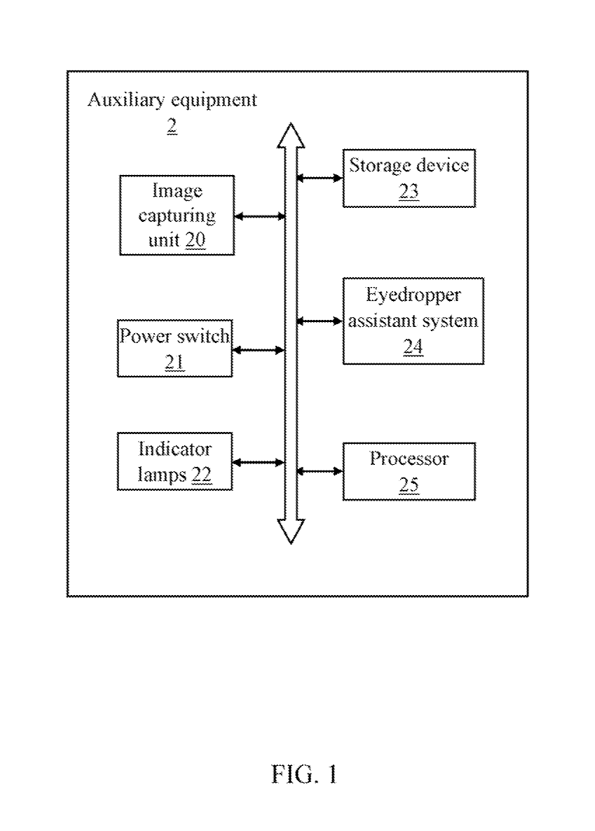

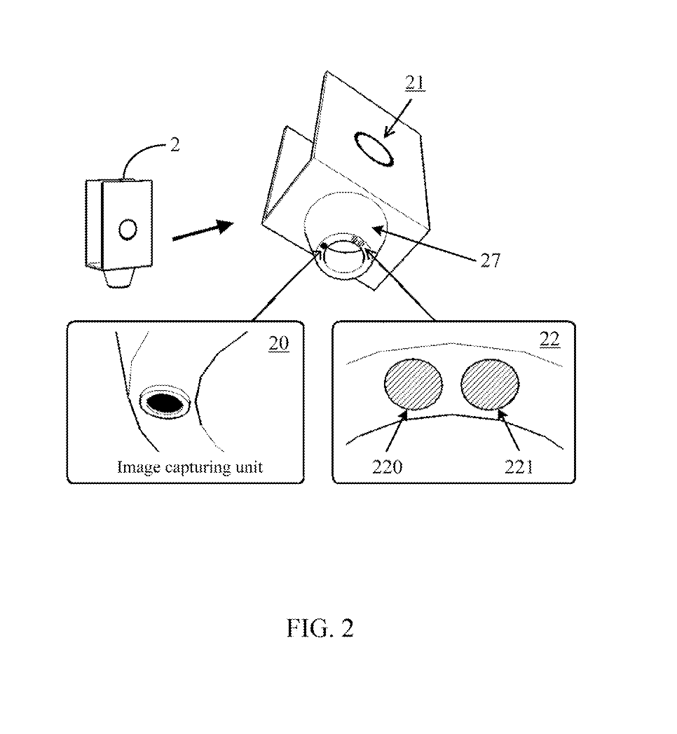

FIG. 1 is a block diagram of one embodiment of an auxiliary equipment 2. The auxiliary equipment 2 may include an image capturing unit 20, a power switch 21, one or more indicator lamps 22, a storage device 23, an eyedropper assistant system 24, and at least one processor 25. FIG. 1 illustrates only one example of the auxiliary equipment 2 that may include more or fewer components than illustrated, or have a different configuration of the various components in other embodiments. A detailed description of the auxiliary equipment 2 is shown in FIGS. 2-4.

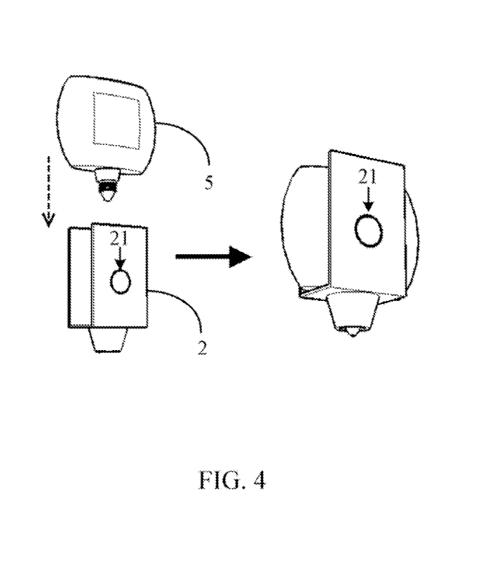

FIG. 2 shows that the image capturing unit 20 and the one or more indicator lamps 22 installed on a front holder 27 of the auxiliary equipment 2. The middle part of the front holder 27 has a hole, the end of the auxiliary equipment 2 is an open shape. An eyedropper (or an eye drop bottle) 5 may be held by the front holder 27 of the auxiliary equipment 2, and a tip of the eyedropper 5 is located in the hole of the front holder 27 (referring to FIG. 4). The power switch 21 is installed on a side wall of the auxiliary equipment 2. In one embodiment, the power switch 21 may be a touch-sensitive switch, such as a capacitive touch-sensitive button. When a conductive object (e.g., a finger of a user) touches the power switch 21, the image capturing unit 20 starts to capture images of the eyes of the user after a preset time interval (e.g., one second), and transmits the captured images to the eyedropper assistant system 24 when the system 24 is started up.

In one embodiment, the one or more indicator lamps 22 include a first indicator lamp 220 and a second indicator lamp 221. The first indicator lamp 220 is activated when the tip of the eyedropper 5 is not located in a proper area of the eye, to prompt the user to adjust a position of the tip of the eyedropper 5. The second indicator lamp 221 is activated when the tip of the eyedropper 5 is located in the proper area of the eye, to prompt the user to use the eyedropper 5.

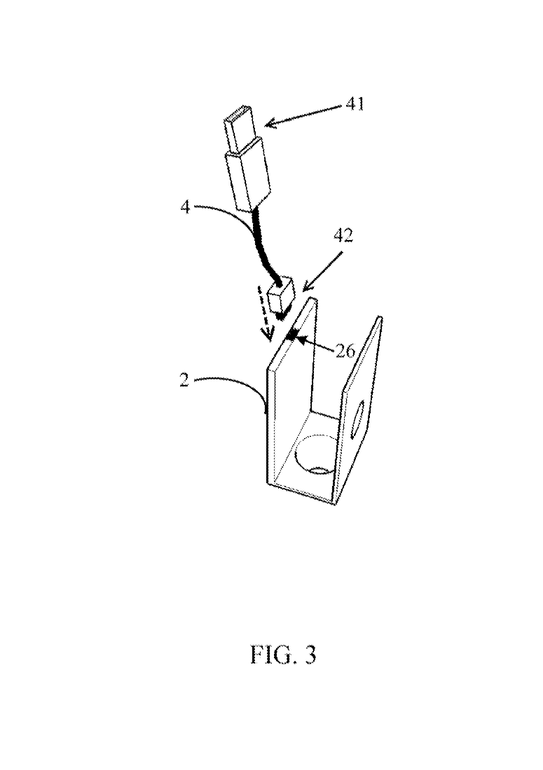

The auxiliary equipment 2 may be connected to other electronic devices (e.g., a computer) through a transmitting line 4, such as a universal serial bus (USB) data bus. The computer may charge up a battery of the auxiliary equipment 2 through the transmitting line 4. For example, as shown in FIG. 3, the transmitting line 4 includes a first connection port (e.g., a USB port) 41 and a second connection port 42. When the first connection port 41 is connected to the computer, and the second connection port 42 is connected to a charge port 26 of the auxiliary equipment 2, the computer starts to charge up the battery of auxiliary equipment 2. The charge port 26 may be installed on a side wall of the auxiliary equipment 2 (referring to FIG. 3).

The eyedropper assistant system 24 detects whether the tip of the eyedropper 5 is located in the proper area of the eye by analyzing the images of the eye captured by the image capturing unit 20, and activates the first indicator lamp 220 when the eyedropper 5 is not located in the proper area of the eye, to prompt the user to adjust the position of the tip of the eyedropper 5. A detailed description is given in the following paragraphs. In one illustrated embodiment, the eyedropper assistant system 24 includes computerized instructions in the form of one or more programs that are executed by the processor 25 and stored in the storage device 23 (or memory). The storage device 23 may be a smart media card, a secure digital card, a compact flash card, or any other memory storage device.

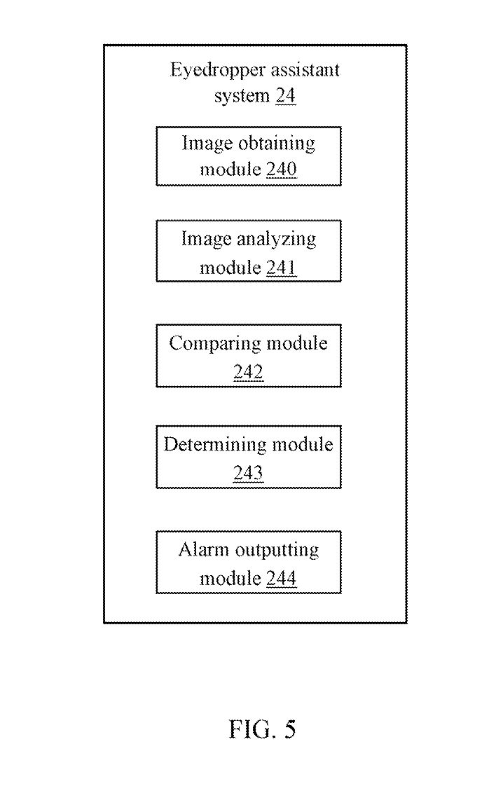

FIG. 5 is a block diagram of function modules of the eyedropper assistant system 24 included in the auxiliary equipment 2. In one embodiment, the eyedropper assistant system 24 may include one or more modules, for example, an image obtaining module 240, an image analyzing module 241, a comparing module 242, a determining module 243, and an alarm outputting module 244. In general, the word “module”, as used herein, refers to logic embodied in hardware or firmware, or to a collection of software instructions, written in a programming language, such as, Java, C, or assembly. One or more software instructions in the modules may be embedded in firmware, such as in an EPROM. The modules described herein may be implemented as either software and/or hardware modules and may be stored in any type of non-transitory computer-readable medium or other storage device. Some non-limiting examples of non-transitory computer-readable medium include CDs, DVDs, BLU-RAY, flash memory, and hard disk drives.

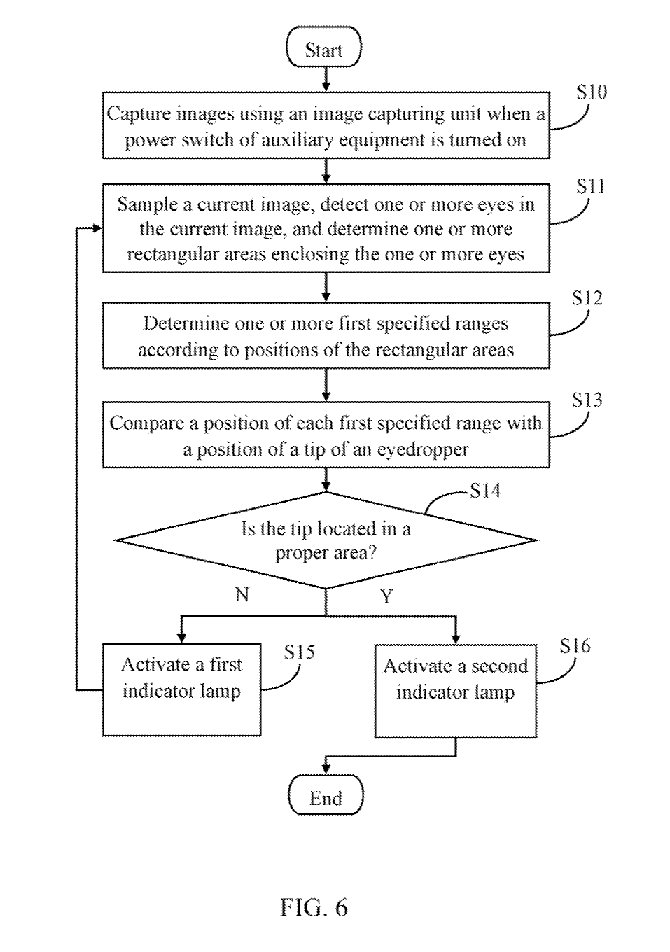

FIG. 6 is a flowchart of one embodiment of a method for assisting with the use of the eyedropper 5 using the auxiliary equipment 2. Depending on the embodiment, additional steps may be added, others removed, and the ordering of the steps may be changed.

In step S10, when the power switch 21 of auxiliary equipment 2 is turned on, the image capturing unit 20 captures images of the eyes of the user at each preset time interval (e.g., one second).

In step S11, the image obtaining module 240 samples a current image captured by the image capturing unit 20. Then, the image analyzing module 241 detects one or more eyes in the current image using an eye detection method, and determines one or more rectangular areas 30 enclosing the one or more eyes. In one embodiment, the eye detection method may be a template matching method which compares the current image with an eyes template to detect the eyes in the current image. For example, as shown in

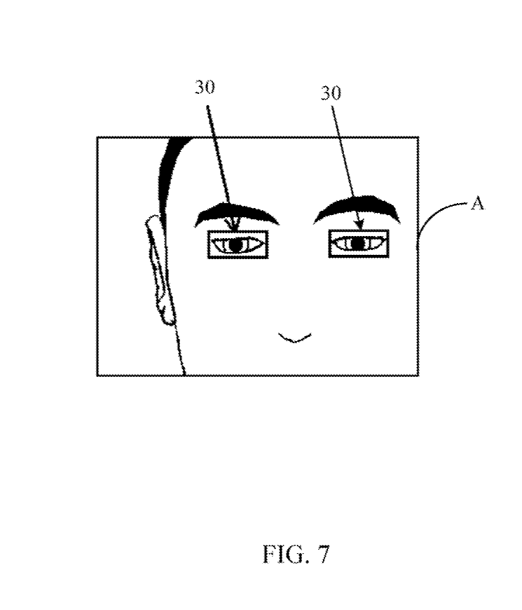

FIG. 7, “A” represents the current image captured by the image capturing unit 20, the rectangular areas 30 are the smallest rectangles enclosing the eyes. Two rectangular areas 30 of two eyes are determined in a normal situation. In other embodiments, the image analyzing module 241 may determine one or more circular areas enclosing the eyes.

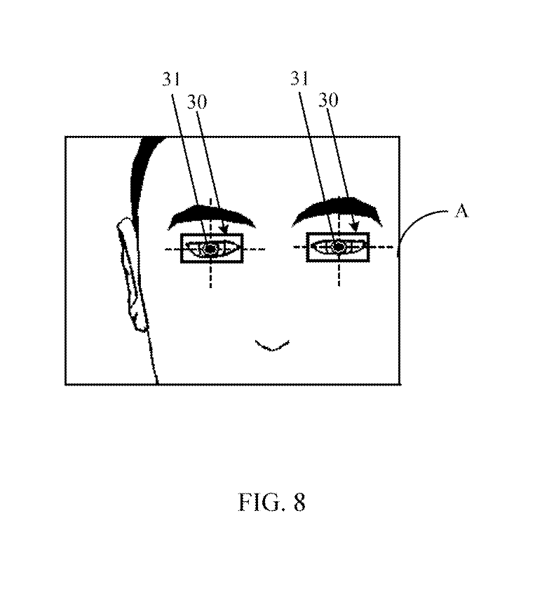

In step S12, the image analyzing module 241 determines one or more first specified ranges 31 of the one or more eyes according to positions of the one or more rectangular areas 30 of the eyes, the first specified ranges 31 are determined to be the proper areas of the eyes for dropping eye drops from the eyedropper 5. Two first specified ranges 31 of two eyes are determined in the normal situation. In one embodiment, the first specified ranges 31 are sub-areas (e.g., circular ranges) of the rectangular areas 30, a center of each rectangular area 30 is determined to be a center of each first specified range 31 (refers to FIG. 8). A radius of each first specified range 31 is equal to a first preset value when the first specified range 31 is a circular range. For example, the radius of each first specified range 31 is equal to a first preset percentage (e.g., 25%) of a width of the rectangular area 30. Suppose that “W” represents the width of each rectangular area 30, “R1” represents the radius of each first specified range 31, then R1=W*25%.

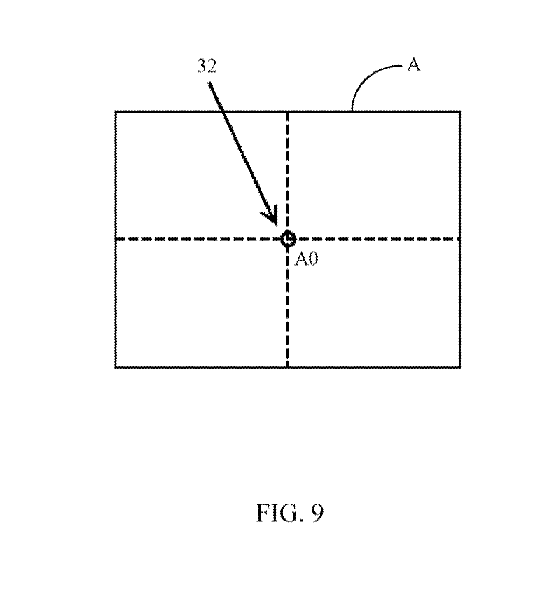

In step S13, the comparing module 242 compares a position of each first specified range 31 with a position of the tip of the eyedropper 5, and determines whether the position of the tip of the eyedropper 5 is located within one of the first specified ranges 31. Because the image capturing unit 20 is installed near the tip of the eyedropper 5, a center of the current image is determined to be the position of the tip of the eyedropper 5. FIG. 9 shows that the comparing module 242 further determines a second specified range (e.g., a circular range) 32 extending from the center “A0” of the current image “A”, and the center “A0” of the current image “A” is determined to be a center of the second specified range 32. A radius of the second specified range 32 is equal to a second preset value when the second specified range 32 is a circular range. For example, the radius of the second specified range 32 is equal to a second preset percentage (e.g., 15%) of a width of the rectangular area 30. Suppose that “W” represents the width of the rectangular area 30, “R2” represents the radius of the second specified range 32, then R2=W*15%.

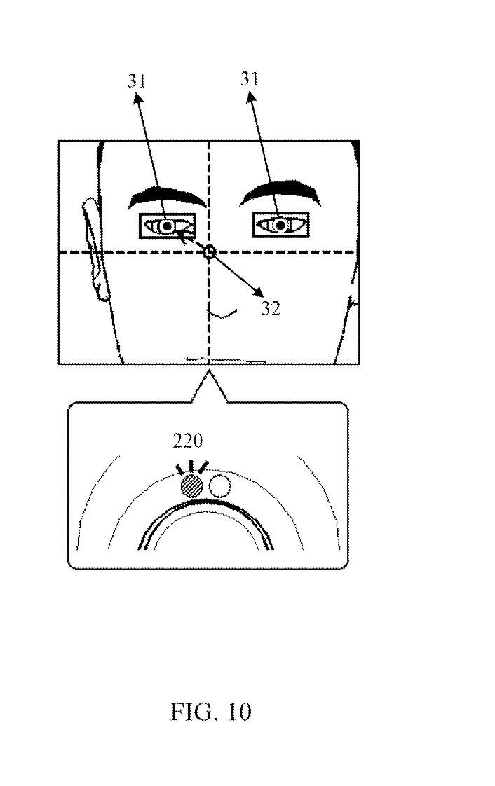

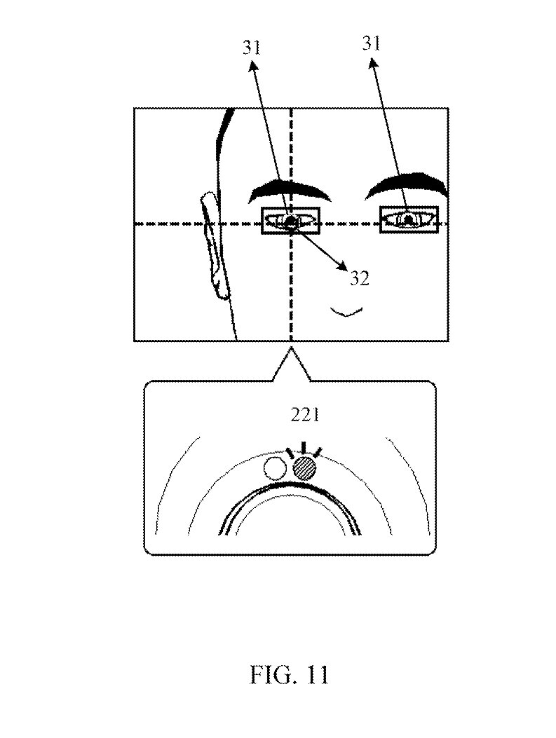

In step S14, the determining module 243 determines whether the tip of the eyedropper 5 is located in one of the proper areas of the eyes by determining whether the position of the tip is located in one of the first specified ranges 31. If all of the first specified ranges 31 are not overlapping with the second specified range 32 (referring to FIG. 10), the determining module 243 determines that the position of the tip of the eyedropper 5 is not located in one of the first specified ranges 31, and is not located in one of the proper areas of the eyes, then the procedure goes to step S15. If one of the first specified ranges 31 is overlapping with the second specified range 32 (referring to FIG. 11), the determining module 243 determines that the position of the tip of the eyedropper 5 is located in one of the first specified ranges 31, and is located in one of the proper areas of the eyes, then the procedure goes to step S16.

In step S15, the alarm outputting module 244 activates the first indicator lamp 220, and controls the first indicator lamp 220 to illuminate with different preset frequencies (referring to FIG. 10), to prompt the user to adjust the position of the tip of the eyedropper 5, then the procedure returns to step S11. A color of the first indicator lamp 220 may be red. In the illustrated embodiment, if a distance between one of the first specified ranges 31 and the second specified range 32 is greater than a preset value (e.g., two centimeters), the alarm outputting module 244 determines that the tip of the eyedropper 5 is away from one of the proper areas of the eyes, and controls the first indicator lamp 220 to illuminate with a lower frequency (e.g., a first preset frequency, such as 500 Hz). If the distance between one of the first specified ranges 31 and the second specified range 32 is less than or equal to the preset value, the alarm outputting module 244 determines that the tip of the eyedropper 5 is closed to one of the proper areas of the eyes, and controls the first indicator lamp 220 to illuminate with a higher frequency (e.g., a second preset frequency, such as 1000 Hz).

In step S16, the alarm outputting module 244 activates the second indicator lamp 221, and controls the second indicator lamp 221 to illuminate with a third preset frequency (e.g., 1500 Hz), to prompt the user to use the eyedropper 5. A color of the second indicator lamp 221 may be green.

In other embodiments, the second indicator lamp 221 may be removed from the auxiliary equipment 2. In this situation, step S16 is executed as follows. The alarm outputting module 244 controls the first indicator lamp 220 to stop illuminating when the tip of the eyedropper 5 is located in one of the proper areas of the eyes, to prompt the user to use the eyedropper 5.

It should be emphasized that the above-described embodiments of the present disclosure, particularly, any embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. An auxiliary equipment, comprising:
a power switch being installed on a side wall of the auxiliary equipment;
an image capturing unit and one or more indicator lamps being installed on a front holder of the auxiliary equipment, a middle part of the front holder having a hole, and an eyedropper being held by the front holder of the auxiliary equipment;
a storage device storing a plurality of images captured by the image capturing unit; and
a processor that samples a current image from the plurality of images when the power switch is turned on, determines whether a tip of the eyedropper is located in one of predetermined areas of eyes of a user by analyzing a position of the tip of the eyedropper in the current image, and activates the one or more indicator lamps according to a determination result, the predetermined areas of the eyes being sub-areas of rectangular areas of the eyes.

2. The auxiliary equipment according to claim 1, further comprising a charge port that is installed on the side wall of the auxiliary equipment.

3. The auxiliary equipment according to claim 1, wherein the one or more indicator lamps comprising a first indicator lamp and a second indicator lamp, the first indicator lamp is activated when the tip of the eyedropper is not located in the predetermined areas of the eyes, and the second indicator lamp is activated when the tip of the eyedropper is located in one of the predetermined areas of the eyes.

4. The auxiliary equipment according to claim 3, wherein the first indicator lamp is activated with different preset frequencies according to a distance between the tip of the eyedropper and one of the rectangular areas of the eyes.

5. The auxiliary equipment according to claim 4, wherein the first indicator lamp is activated with a first preset frequency when the distance is greater than a preset value, and the first indicator lamp is activated with a second preset frequency when the distance is less than or equal to the preset value, the second preset frequency being greater than the first preset frequency.

6. A method for assisting usage of an eyedropper, the method comprising:
providing an auxiliary equipment comprising an image capturing unit, a power switch, a first indicator lamp, and a second indicator lamp;
sampling a current image captured by the image capturing unit when the power switch is turn on;
detecting one or more eyes in the current image using an eye detection method, and determining one or more rectangular areas enclosing the one or more eyes;
determining one or more first specified ranges according to positions of the one or more rectangular areas, the first specified ranges being determined to be proper areas of the eyes for dropping eye drops from the eyedropper;
comparing a position of each of the first specified ranges with a position of a second specified range of a tip of the eyedropper;
determining whether the second specified range is overlapping with one of the first specified ranges;
activating the first indicator lamp when all of the first specified ranges are not overlapping with the second specified range; and
activating the second indicator lamp when one of the first specified ranges is overlapping with the second specified range.

7. The method according to claim 6, wherein the first specified ranges are sub-areas of the rectangular areas, and a center of each of the rectangular areas is determined to be a center of each of the first specified ranges, the second specified range is extended from a center of the current image, and the center of the current image is determined to be a center of the second specified range.

8. The method according to claim 6, further comprising: activating the first indicator lamp with different preset frequencies according to a distance between the second specified range and one of the first specified ranges.

9. The method according to claim 8, further comprising: activating the first indicator lamp with a first preset frequency when the distance is greater than a preset value.

10. The method according to claim 9, further comprising: activating the first indicator lamp with a second preset frequency when the distance is less than or equal to the preset value, the second preset frequency being greater than the first preset frequency.

11. A non-transitory storage medium having stored thereon instructions that, when executed by a processor of an auxiliary equipment, causes the auxiliary equipment to perform a method for assisting usage of an eyedropper using the auxiliary equipment, the method comprising:
sampling a current image captured by an image capturing unit of the auxiliary equipment when a power switch of the auxiliary equipment is turn on;
detecting one or more eyes in the current image using an eye detection method, and determining one or more rectangular areas enclosing the one or more eyes;
determining one or more first specified ranges according to positions of the one or more rectangular areas, the first specified ranges being determined to be proper areas of the eyes for dropping eye drops from the eyedropper;
comparing a position of each of the first specified ranges with a position of a second specified range of a tip of the eyedropper;
determining whether the second specified range is overlapping with one of the first specified ranges;
activating a first indicator lamp of the auxiliary equipment when all of the first specified ranges are not overlapping with the second specified range; and
activating a second indicator lamp of the auxiliary equipment when one of the first specified ranges is overlapping with the second specified range.

12. The non-transitory storage medium according to claim 11, wherein the first specified ranges are sub-areas of the rectangular areas, and a center of each of the rectangular areas is determined to be a center of each of the first specified ranges, the second specified range is extended from a center of the current image, and the center of the current image is determined to be a center of the second specified range.

13. The non-transitory storage medium according to claim 11, wherein the method further comprises: activating the first indicator lamp with different preset frequencies according to a distance between the second specified range and one of the first specified ranges.

14. The non-transitory storage medium according to claim 13, wherein the method further comprises: activating the first indicator lamp with a first preset frequency when the distance is greater than a preset value.

15. The non-transitory storage medium according to claim 14, wherein the method further comprises: activating the first indicator lamp with a second preset frequency when the distance is less than or equal to the preset value, the second preset frequency being greater than the first preset frequency.
